(19) United States
(12) Patent Application Publication
(10) Pub. No.: US 2004/0220646 A1
(43) Pub. Date: Nov. 4, 2004

(54) Mattress

(76) Inventor: Mu-Hyun Cho, Seoul (KR)

Correspondence Address: INTELLECTUAL PROPERTY LAW GROUP LLP, 12 SOUTH FIRST STREET, SUITE 1205, SAN JOSE, CA, 95113, US

(21) Appl. No.: 10/833,906

(22) Filed: Apr. 27, 2004

(30) Foreign Application Priority Data
Apr. 29, 2003 (KR) 10-2003-0027251

(51) Int. Cl. A61F 7/00; A61F 7/12; A61F 2/00

(52) U.S. Cl. 607/96

(57) ABSTRACT

A mattress having a thermal device, in which a variety of far-infrared radiation materials are arrayed in a laminated unit of the mattress to define various patterns, thus allowing a user to optimally absorb the far-infrared radiation while resting. The mattress includes an electric power supply unit, the laminated unit, and a support unit. The laminated unit comprises a thermal plate to generate heat by electricity supplied from the electric power supply unit, a far-infrared unit housing placed on the thermal plate with a variety of far-infrared radiation materials of a powdered-phase or a solid-phase arrayed in the far-infrared unit housing to form various patterns, and a finishing material sheet placed on the far-infrared unit housing. The support unit supports the laminated unit while integrating the thermal plate, the far-infrared unit housing and the finishing material sheet into a single body. The laminated unit integrated by the support unit is seated into a seating depression of a mattress body having a temperature control unit.

### BACKGROUND OF THE INVENTION

1. Field of the Invention

The present invention relates, in general, to a mattress used as a cushion and, more particularly, to a structure of a mattress having a thermal device, in which a variety of far-infrared radiation materials are arrayed in a laminated unit of the mattress to define various patterns, thus allowing a user to optimally absorb the far-infrared radiation while resting.

2. Description of the Prior Art

Recently, some mattresses have been developed and proposed as health equipment to provide an effect that promotes the health of users while lying thereon, as well as provide a typical function thereof to serve as comfortable mattresses which support the users while resting.

In a detailed description, a magnetic mattress, with a plurality of magnets arranged in the mattress to provide an effect of promoting health of users by magnetic fields generated by the magnets, has been proposed and used. Furthermore, a far-infrared mattress provided therein with far-infrared radiation materials, such as jade, charcoal, elvan, loess and the like, to promote blood circulation and the metabolism in user's body by the far-infrared radiation of the materials has been proposed.

However, conventional magnetic or far-infrared mattresses are problematic in that the mattresses must be used only in a state of being placed such that a surface with the magnets or the jade, charcoal, elvan, loess and the like is directed upward to support the user's body thereon.

Furthermore, conventional mattresses force a user, who wants to take the magnetic effect of magnets and the far-infrared radiation effect of jade, charcoal, elvan, loess and the like, to purchase a magnetic mattress and a far-infrared mattress, separately.

### SUMMARY OF THE INVENTION

Accordingly, the present invention has been made keeping in mind the above problems occurring in the prior art, and an object of the present invention is to provide a mattress having a thermal device, in which a variety of far-infrared radiation materials are arrayed in a laminated unit of the mattress to define various patterns, thus allowing a user to optimally absorb the far-infrared radiation while resting.

Another object of the present invention is to provide a mattress having a thermal device, which is reduced by weight and production costs thereof, while providing better effects on a user's body in comparison with conventional mattresses.

A further object of the present invention is to provide a mattress having a thermal device, which is produced through a simple process to enhance work efficiency and reduce time consumption while producing the mattress.

In order to achieve the above objects, an embodiment of the present invention provides a mattress, comprising: an electric power supply unit; a laminated unit comprising a thermal plate to generate heat by electricity supplied from the electric power supply unit, a far-infrared unit housing placed on the thermal plate with a variety of far-infrared radiation materials of a powdered-phase or a solid-phase arrayed in the far-infrared unit housing to form various patterns, and a finishing material sheet placed on the far-infrared unit housing; and a support unit to support the laminated unit while integrating the thermal plate, the far-infrared unit housing and the finishing material sheet into a single body; wherein the laminated unit integrated by the support unit is set into a seating depression of a mattress body having a temperature control unit.

In another embodiment of the present invention, the far-infrared radiation materials may comprise of two or more materials selected from the group consisting of charcoal, jade, loess, elvan and quartz.

In an embodiment of the present invention, the far-infrared unit housing may comprise a box-shaped body, with the variety of far-infrared radiation materials irregularly arrayed in the box-shaped body.

In another embodiment of the present invention, the far-infrared unit housing may comprise a box-shaped body, with the variety of far-infrared radiation materials arrayed in the box-shaped body to respectively define a plurality of lanes in the box-shaped body.

In another embodiment of the present invention, the far-infrared unit housing may comprise a box-shaped body, with a plurality of latitudinal and longitudinal partitions arrayed in the box-shaped body to define a plurality of cells which respectively contain the variety of far-infrared radiation materials therein.

In yet another embodiment of the present invention, the far-infrared radiation materials of the solid-phase may comprise raw stones.

The mattress of an embodiment of the present invention may further comprise a cushion sheet combined with a function of shielding electromagnetic waves, and interposed between the far-infrared unit housing and the finishing material sheet.

In order to achieve the above objects, an embodiment of the present invention also provides a mattress, comprising: an electric power supply unit; a laminated unit comprising a thermal plate to generate heat by electricity supplied from the electric power supply unit, a fixing panel placed on the thermal plate with a variety of far-infrared radiation materials of a solid-phase arrayed on the fixing panel to form various patterns, and a finishing material sheet placed on the fixing panel; and a support unit to support the laminated unit while integrating the thermal plate, the fixing panel with the far-infrared radiation materials, and the finishing material sheet into a single body; wherein the laminated unit integrated by the support unit is set into a seating depression of a mattress body having a temperature control unit.

In the embodiment of the present invention, the far-infrared radiation materials of the solid-phase arrayed on the fixing panel may be integrated into a single body by an adhesive means.

The mattress may further comprise a cushion sheet combined with a function of shielding electromagnetic waves, and interposed between the fixing panel and the finishing material sheet.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view of a mattress according to a first embodiment of the present invention;

FIG. 2 is a partially-broken perspective view of the mattress according to the first embodiment of the present invention;

FIG. 3 is a sectional view illustrating a far-infrared unit housing installed in the mattress according to the first embodiment of the present invention;

FIGS. 4a through 5d are plan views respectively illustrating a plurality of far-infrared unit housings of various patterns installed in the mattress, according to different modifications of the first embodiment of the present invention;

FIG. 6 is a partially-broken perspective view of a mattress according to a second embodiment of the present invention; and

FIG. 7 is a sectional view illustrating a variety of far-infrared radiation materials of a solid-phase arrayed on a fixing panel of the mattress according to the second embodiment of the present invention.

### DETAILED DESCRIPTION OF SPECIFIC EMBODIMENTS

Reference will now be made in greater detail to a preferred embodiment of the invention, an example of which is illustrated in the accompanying drawings. Wherever possible, the same reference numerals will be used throughout the drawings and the description to refer to the same or like parts.

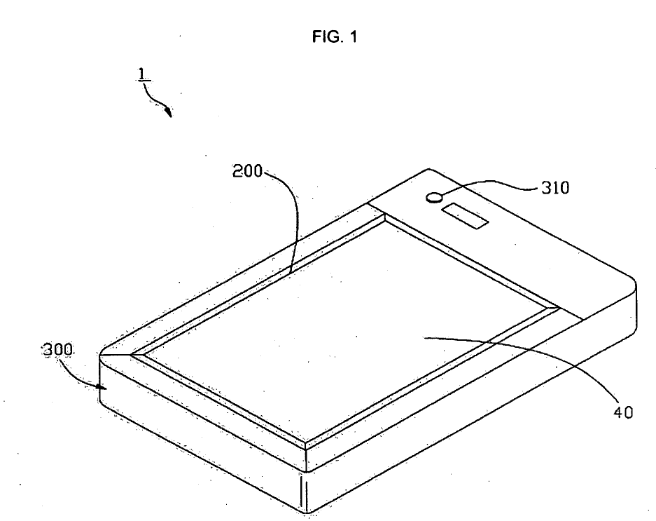

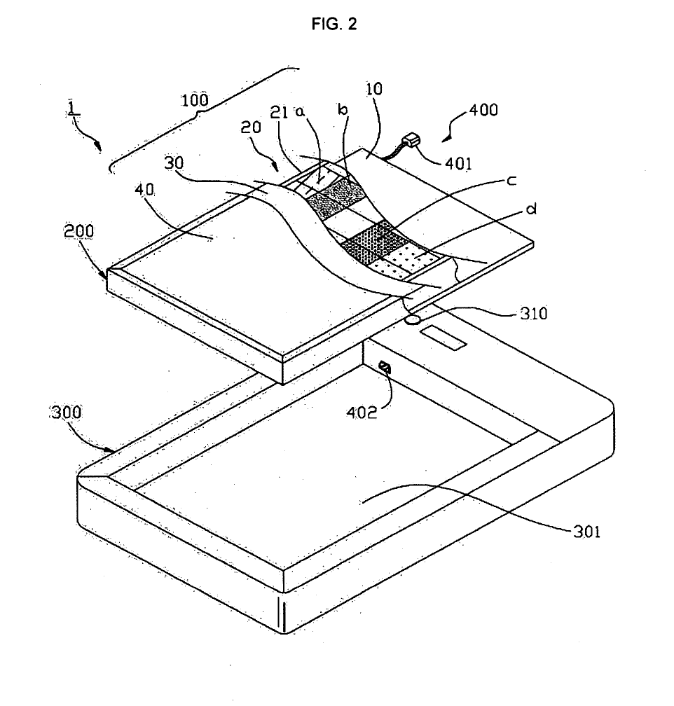

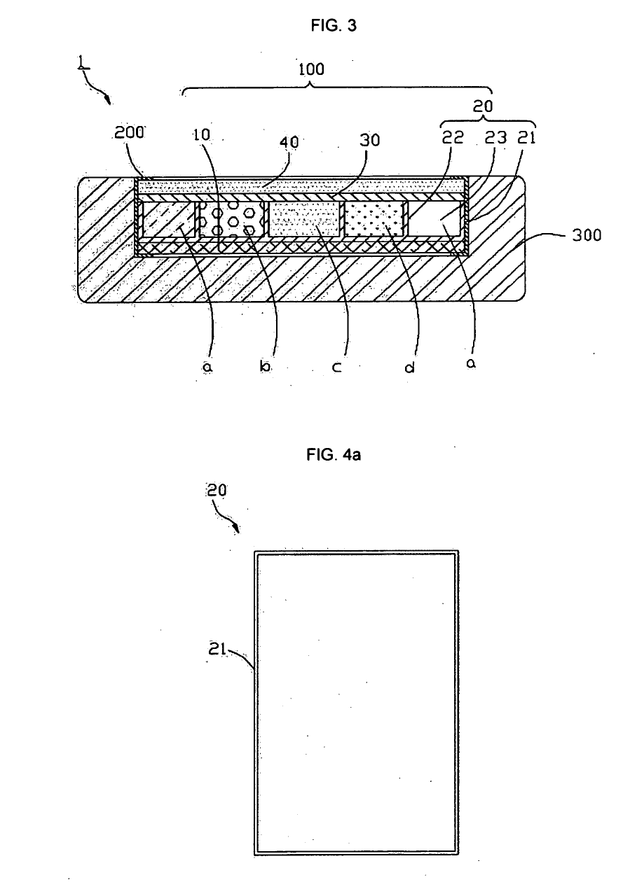

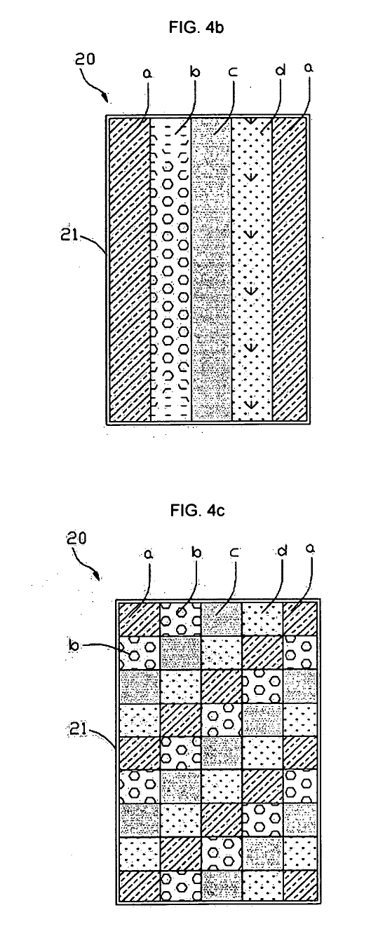

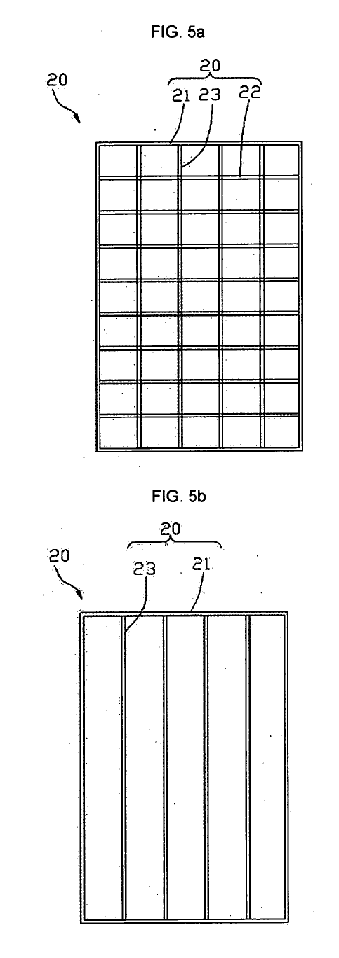

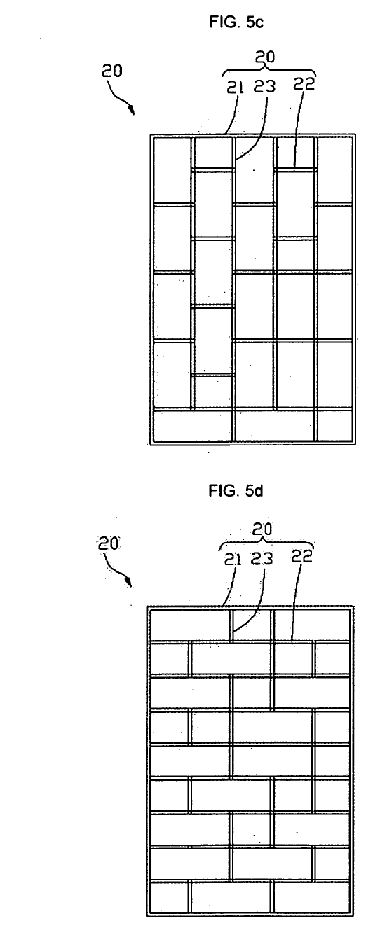

FIG. 1 is a perspective view of a mattress according to a first embodiment of the present invention. FIG. 2 is a partially-broken perspective view of the mattress according to the first embodiment of the present invention. FIG. 3 is a sectional view illustrating a far-infrared unit housing installed in the mattress according to the first embodiment of the present invention. FIGS. 4a through 5d are plan views respectively illustrating a plurality of far-infrared unit housings of various patterns installed in the mattress, according to different modifications of the first embodiment of the present invention.

As shown in FIGS. 1 and 2, the mattress 1 according to the first embodiment of the present invention comprises an electric power supply unit 400, a laminated unit 100, and a support unit 200 to support the laminated unit 100 while integrating the elements of the laminated unit 100 into a single body, and the laminated unit 100 integrated by the support unit 200 is set into a seating depression 301 of a mattress body 300 having a temperature control unit 310.

The laminated unit 100 comprises a thermal plate 10 to generate heat by electricity supplied from the electric power supply unit 400, and a far-infrared unit housing 20 placed on the thermal plate 10, with a variety of far-infrared radiation materials of a powdered-phase or a solid-phase arrayed in the far-infrared unit housing 20 to form various patterns. The laminated unit 100 further comprises a cushion sheet 30 combined with a function of shielding electromagnetic waves, and placed on the far-infrared unit housing 20. The laminated unit 100 further comprises a finishing material sheet 40 placed on the cushion sheet 30.

The far-infrared unit housing 20 comprises a box-shaped body 21 having a rectangular shape as shown in FIG. 4a.

In the first embodiment of the present invention, the raw stones of a variety of far-infrared radiation materials, which include charcoal (a), jade (b), loess (c), elvan (d) and quartz (not shown) such as amethyst, may be arrayed in the far-infrared unit housing 20 to respectively define a plurality of lanes in the housing 20, as shown in FIG. 4b. Alternatively, the variety of far-infrared radiation materials, which include the charcoal (a), jade (b), loess (c), elvan (d) and quartz (not shown) such as amethyst, may be alternately arrayed in the far-infrared unit housing 20 to form checks, as shown in FIG. 4c.

In the embodiment of the present invention, the variety of far-infrared radiation materials contained in the far-infrared unit housing 20 may comprise all the above-mentioned charcoal, jade, loess, elvan and quartz such as amethyst, or comprise two or more materials selected from the group consisting of the above-mentioned materials.

Furthermore, the far-infrared unit housing 20 may comprise a plurality of latitudinal partitions 22 and/or a plurality of longitudinal partitions 23 arrayed in the box-shaped body 21 to define a plurality of cells in the box-shaped body 21, as shown in FIGS. 3, 5a through 5d.

The charcoal, contained in the far-infrared unit housing 20, particularly, charcoal made of oak wood, allows persons suffering insomnia caused by accumulated stress fatigue, for example, businessmen or students, or persons with neurological disorder, to rest or sleep comfortably. Furthermore, the charcoal absorbs sweat and heat generated from the body of a user, and discharges anions to allow the user to have a healthful rest and sleep. Thus, in the present invention, the charcoal may be preferably used with both raw stones of jade (b) and loess (c) in the far-infrared unit housing 20 at an amount ratio of 1:1:1. It is also preferable that the charcoal is contained in the far-infrared radiation materials in an amount of 30% to 40% by weight based on the total weight of the far-infrared radiation materials.

In the first embodiment of the present invention, the plurality of latitudinal and longitudinal partitions 22 and 23 may be arrayed in the box-shaped body 21 to define regular cells in the body 21, as shown in FIGS. 5a and 5b. Alternatively, the plurality of latitudinal and longitudinal partitions 22 and 23 may be arrayed in the box-shaped body 21 to define irregular cells in the body 21, as shown in FIGS. 5c and 5d.

Furthermore, either the latitudinal partitions 22 or the longitudinal partitions 23 may be exclusively arrayed in the box-shaped body 21 at regular intervals, as shown in FIG. 5b.

Furthermore, the finishing material sheet 40 may comprise of a rock sheet, a quartz sheet, a cork sheet, a jade sheet, a loess sheet, a ceramic sheet, etc. The cushion sheet 30 combined with the function of shielding the electromagnetic waves include silver, thus effectively killing MRSA (Methicillin Resistant Staphylococcus Aureus) which is a pathogen having high tolerance, up to 99.9%, as well as killing Staphylococcus, Pneumococcus, Pseudomonas, Coliform bacilli, etc. Furthermore, the above-mentioned cushion sheet 30, including the silver, provides a hygienic effect and removes odor.

Furthermore, the cushion sheet 30 including the silver radiates far-infrared rays, which are beneficial for the health of the users. That is, the far-infrared rays promote water molecular vibrations in the user's body, thus promoting blood circulation and the metabolism of the user's body.

The electric power supply unit 400 that supplies electricity to the thermal plate 10 may be fabricated by directly connecting the thermal plate 10 to the temperature control unit 310 of the mattress body 300 via an electric wire. Alternatively, the electric power supply unit 400 may be fabricated by providing both a first connector 401 on the thermal plate 10 and a second connector 402 in the seating depression 301 of the mattress body 300 as shown in FIG. 2, so that the first and second connectors 401 and 402 are connected to each other.

In the above case, the second connector 402 is connected to the temperature control unit 310 of the mattress body 300, while the temperature control unit 310 is connected to a plug (not shown).

To produce the mattress 1 having the above-mentioned construction according to the first embodiment of the present invention, the laminated unit 100 integrated by the support unit 200 is produced, prior to being set into the seating depression 301 of the mattress body 300. That is, the thermal plate 10 used as a thermal device to generate heat by electricity supplied from an external electric power source through the electric power supply unit 400 is first positioned. Thereafter, the far-infrared unit housing 20, with the variety of far-infrared radiation materials of the powdered-phase or the solid-phase arrayed in the far-infrared unit housing 20 to form various patterns, is placed on the thermal plate 10. Thereafter, the cushion sheet 30 combined with the function of shielding the electromagnetic waves is placed on the far-infrared unit housing 20. The finishing material sheet 40 is, thereafter, placed on the cushion sheet 30, thus providing the laminated unit 100. After sequentially layering the elements of the laminated unit 100 as described above, the elements of the laminated unit 100 are integrated into a single body by the support unit 200 which is a framework having a U-shaped cross-section. The laminated unit. 100 integrated by the support unit 200 is, thereafter, set into the seating depression 301 of the mattress body 300 having the temperature control unit 310 as shown in FIG. 2, thus providing a desired mattress having a thermal device.

In the above state, the first connector 401 provided on the thermal plate 10 is connected to the second connector 402 provided in the seating depression 301 of the mattress body 300. Thus, the thermal plate 10 can be turned on by electricity supplied from an external electric power source through the first and second connectors 401 and 402.

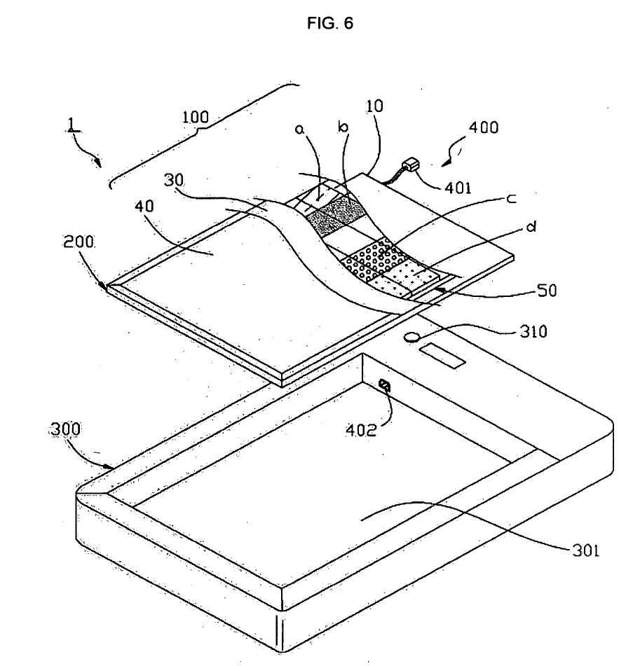

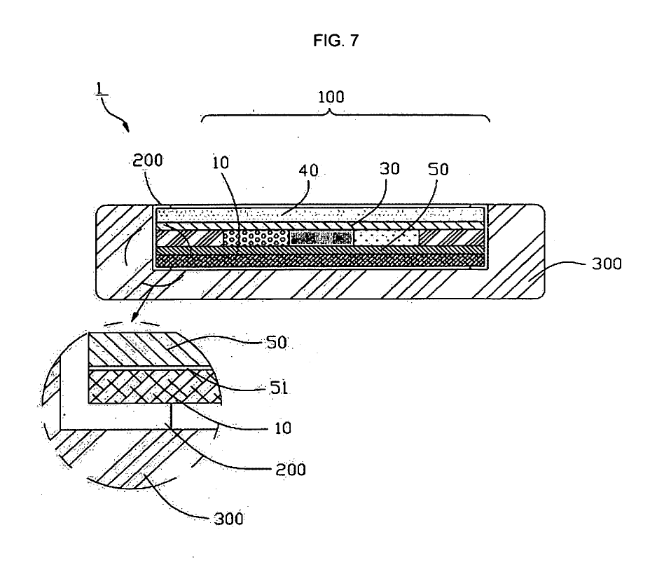

FIG. 6 is a partially-broken perspective view of a mattress according to a second embodiment of the present invention. FIG. 7 is a sectional view illustrating a variety of far-infrared radiation materials of a solid-phase arrayed on a fixing panel of the mattress according to the second embodiment of the present invention.

In the second embodiment of the present invention, the general shape of the mattress remains the same as that of the first embodiment, and thus, those elements similar to the first and second embodiments carry the same reference numerals and further explanation is hot deemed necessary.

As shown in FIGS. 6 and 7, the mattress 1 according to the second embodiment of the present invention also comprises an electric power supply unit 400, a laminated unit 100, and a support unit 200 to support the laminated unit 100 while integrating the elements of the laminated unit 100 into a single body, and the laminated unit 100 integrated by the support unit 200 is set into a seating depression 301 of a mattress body 300 having a temperature control unit 310.

The laminated unit 100 comprises a thermal plate 10 to generate heat by electricity supplied from the electric power supply unit 400, and a fixing panel 50 placed on the thermal plate 10, with a variety of far-infrared radiation materials of a solid-phase arrayed on the fixing panel 50 to form various patterns. The laminated unit 20 further comprises a cushion sheet 30 combined with a function of shielding electromagnetic waves, and placed on the fixing panel 50. The laminated unit 20 further comprises a finishing material sheet 40 placed on the cushion sheet 30.

In the second embodiment, the variety of far-infrared radiation materials of the solid-phase may be directly arrayed on the fixing panel 50 to form the various patterns without being integrated. Alternatively, the far-infrared radiation materials of the solid-phase arrayed on the fixing panel 50 to form the various patterns may be integrated into a single body by an adhesive means 51 as shown in FIG. 7.

As apparent from the above description, the present invention provides a mattress having a thermal device, in which a variety of far-infrared radiation materials are arrayed in a far-infrared unit housing or on a fixing panel of a laminated unit of the mattress to form a regular or irregular pattern. Thus, the mattress is advantageous in that a user optimally absorbs the far-infrared radiation from the mattress. Another advantage of the mattress according to the present invention resides in that the mattress is easily produced.

Although preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A mattress, comprising:
an electric power supply unit;
a laminated unit comprising:
a thermal plate to generate heat by electricity supplied from the electric power supply unit;
a far-infrared unit housing placed on the thermal plate, with a variety of far-infrared radiation materials of a powdered-phase or a solid-phase arrayed in the far-infrared unit housing to form various patterns; and
a finishing material sheet placed on the far-infrared unit housing; and
a support unit to support the laminated unit while integrating the thermal plate, the far-infrared unit housing and the finishing material sheet into a single body;
wherein the laminated unit integrated by the support unit is set into a seating depression of a mattress body having a temperature control unit.

2. The mattress according to claim 1, wherein the far-infrared radiation materials comprise two or more materials selected from the group consisting of charcoal, jade, loess, elvan and quartz.

3. The mattress according to claim 1 or 2, wherein the far-infrared unit housing comprises a box-shaped body, with the variety of far-infrared radiation materials irregularly arrayed in the box-shaped body.

4. The mattress according to claim 1 or 2, wherein the far-infrared unit housing comprises a box-shaped body, with the variety of far-infrared radiation materials arrayed in the box-shaped body to respectively define a plurality of lanes in the box-shaped body.

5. The mattress according to claim 1 or 2, wherein the far-infrared unit housing comprises a box-shaped body, with a plurality of latitudinal and longitudinal partitions arrayed in the box-shaped body to define a plurality of cells which respectively contain the variety of far-infrared radiation materials therein.

6. The mattress according to claim 1, wherein the far-infrared radiation materials of the solid-phase comprise raw stones.

7. The mattress according to claim 1, further comprising a cushion sheet combined with a function of shielding electromagnetic waves, and interposed between the far-infrared unit housing and the finishing material sheet.

8. A mattress, comprising:
an electric power supply unit;
a laminated unit comprising:
a thermal plate to generate heat by electricity supplied from the electric power supply unit;
a fixing panel placed on the thermal plate, with a variety of far-infrared radiation materials of a solid-phase arrayed on the fixing panel to form various patterns; and
a finishing material sheet placed on the fixing panel; and
a support unit to support the laminated unit while integrating the thermal plate, the fixing panel with the far-infrared radiation materials, and the finishing material sheet into a single body;
wherein the laminated unit integrated by the support unit is set into a seating depression of a mattress body having a temperature control unit.

9. The mattress according to claim 8, wherein the far-infrared radiation materials comprise two or more materials selected from the group consisting of charcoal, jade, loess, elvan and quartz.

10. The mattress according to claim 8, wherein the far-infrared radiation materials of the solid-phase arrayed on the fixing panel are integrated into a single body by adhesive means.

11. The mattress according to claim 8, further comprising a cushion sheet combined with a function of shielding electromagnetic waves, and interposed between the fixing panel and the finishing material sheet.
